(19) United States
(12) Patent Application Publication
(10) Pub. No.: US 2004/0036840 A1
(43) Pub. Date: Feb. 26, 2004

(54) Apparatus and method for testing visual acuity and fixation control

(76) Inventors: Joseph A Marino, Park Ridge, IL (US); Steven B Nordstrom, Hinsdale, IL (US)

Correspondence Address: Timothy E Nauman, Fay Sharpe Fagan Minnich & McKee, 7th Floor, 1100 Superior Avenue, Cleveland, OH, 44114-2518, US

(21) Appl. No.: 10/398,314

(22) PCT Filed: Mar. 21, 2002
(86) PCT No.: PCT/US02/08824

(51) Int. Cl. A61B 3/02

(52) U.S. Cl. 351/239

(57) ABSTRACT

A method and apparatus for conducting a visual acuity test are provided. The method includes displaying a visual acuity test on a display device (20) that is initiated by a controller (16) such as a handheld keypad controller (16), which is operatively associated with the computer-processing, unit (12). The patient's eye is fixated using an eye fixation assessment on a display device during portions of the visual acuity test and is initiated by a controller such as a foot pedal controller (14), which is operatively associated with the CPU. Contrast sensitivity can also he evaluated on the display device by selectively altering the contrast of an image and background on the display device (20).

### CLAIM OF PRIORITY

This application claims priority from U.S. Provisional Application Serial No. 60/277,691, filed on Mar. 21, 2001.

### FIELD OF THE INVENTION

The present invention relates to assessing visual acuity. It finds particular application in conjunction with testing a patient in an ophthalmology examination to determine certain astigmatisms, eye diseases and disorders. However, it is to be appreciated that the present invention is also amenable to other like applications.

### BACKGROUND OF THE INVENTION

In an ophthalmology examination, a health care professional must complete a broad range of tests in order to assess visual acuity, including the presence of astigmatisms, eye diseases and disorders. A loss of visual acuity or sharpness is commonly referred to as blurred vision, and results in unclear visual details. Vision loss is the inability to perceive visual stimuli, which is a significant threat to the quality of life. Therefore, a visual acuity test is very important in detecting changes in vision and loss of vision.

A visual acuity test (VAT) measures the distance a patient stands from an eye chart, which is typically mounted on a wall, and measures the smallest line of text that the patient is able to read clearly. A typical VAT chart includes a large single letter at the top, followed by rows of alphanumeric characters (e.g., letters) underneath continuing to the bottom of the chart, where each subsequent row will typically be of decreasing size for a patient to read aloud. The test is usually conducted at the office of a health care professional who will ask the patient to sit or stand about twenty feet from the VAT chart on an opposing wall. The patient is instructed to remove any contact lenses, eyeglasses, and all other corrective vision apparatus. The health care professional covers one eye of the patient while the patient attempts to read the VAT chart, and the patient continues to read down the chart to smaller and smaller letters until the patient is no longer able to read the letters because the letters appear too blurry.

The VAT provides the health care professional with important information about vision clarity. The result of the VAT is expressed as a fraction. The top number refers to the distance the patient stands from the chart, which is typically about twenty feet. The bottom number indicates the distance at which a person with normal eyesight could read the line with the smallest letters that the patient could correctly read.

More recently health care professionals use one of two systems for the completion of VATs. The first device includes a remote display device, a computer processing unit (CPU) and hand-controlled, illuminated, wired, remote control keypad with a liquid crystal display (LCD). The display device is typically a monitor, and is used to display eye charts, pictures and various other optotypes—all of which are standardized for use in VAT. The basic design of this hand-operated controller requires a proliferation of very small keys to accommodate all of the provided tests and test modes, while also requiring numerous keystrokes to initiate the desired display. This makes this particular system difficult to learn and cumbersome to operate. In addition to the difficulties of learning and operating this system, physicians utilizing these handheld controllers have reported considerable unit failures within five years. Also, they have an inability to accurately use this system for visual acuity tests in small exam rooms (less than ten feet). In shorter rooms, the method of character generation used precludes either accurately sizing the various optotypes or accurately assembling the characters.

The second approach to administering a VAT utilizes a wireless, non-illuminated, remote control device. The health care professional again presses a series of keys to display the acuity images on a monitor. In addition to the complexities of numerous keystrokes to initiate testing and the large number of acuity tests, as well as the small exam room miscalibration, another problem exists with this technology. This system requires the health care professional to turn away from the patient and precisely point the remote control device at the monitor. In addition, since this system does not include an LCD display, the health care professional does not know what test is coming up until it appears on the monitor. This has been found to be inconvenient, more prone to error and more time-consuming for the health care professional.

With respect to both approaches, despite having a large number of available visual displays, there are only a few pictures to select from. This can make it more difficult to properly examine patients across different socioeconomic and cultural backgrounds, since the patient may not be familiar with the few images available for display. In addition, with a specific number of test charts, pictures and optotypes on each keypad, specific test modes and sizes cannot be accommodated as desired by the health care professional. Thus, existing systems do not allow custom programming of the tests or optotypes on a VAT system.

Furthermore, when testing a patient's ability to detect contrast, or contrast sensitivity, a health care professional typically tests only the ability to see black and white “low contrast” using gray images printed on planar material (e.g., paper, cardboard or plastic). These images are usually held up by the health care professional and the patient then describes the images using a reflected light source. A similar method uses translucent plastic overprinted with characters of varying density through which a light is projected. These methods have numerous issues, including the possibility to test inconsistently as light levels change in the room and the inability to test contrast sensitivity in anything other than a few shades of black and white. Further, due to the use of standard printed materials, few densities can be used accurately, which makes an early detection of contrast sensitivity changes in the patient difficult to detect. Also, these methods will typically require more than one tester to conduct the test or a single health care professional must stop and start testing to change different density charts, thus becoming cumbersome to the health care professional.

As previously mentioned, the health care professional also examines patient's eyes for astigmatisms, disease and other eye disorders. It is critical in these examinations for the health care professional to get the patient to fixate all eye movement, except at the direction of the health care professional. This has proven to be extremely difficult for children. To respond to this need for eye fixation, a system is used comprising a monitor, video or DVD player and battery operated moving toy animals all housed in an open cabinet that the patient faces while the health care professional actuates a foot pedal to operate the toys and/or video. For the several seconds that the toy moves and the video plays, the patient will fix their eyes on the target long enough to complete the exam. With this current approach, many different pieces of equipment are required and this system is separate from the VAT. This also takes up valuable space in the examination room of the health care professional.

Therefore, it would be particularly desirable to provide a new method and apparatus for completing a VAT that includes eye fixation and the ability to test contrast sensitivity that overcomes the above-described problems and inconveniences.

### SUMMARY OF THE INVENTION

A method for testing the visual acuity of the patient's eyesight is provided. The method includes displaying a VAT on a display device, such as a monitor. The VAT is initiated by a controller, such as a handheld keypad controller, which is operatively associated with a computer-processing unit (CPU). The method further includes use of an eye fixation assessment on a display device during portions of the VAT. Eye fixation can be initiated by a foot pedal controller which is operatively associated with the CPU. In using the visual acuity and fixation controlled software, the visual acuity of the patient can be determined.

In a preferred embodiment, a VAT apparatus according to the present invention includes a CPU with an associated display device. The VAT apparatus further includes a handheld keypad controller that is operatively associated with the CPU, the keypad controller having a liquid crystal display. Furthermore, the visual acuity testing apparatus according to the present invention includes a foot pedal controller that is operatively associated with the CPU. It is understood that other input devices can be-utilized in this invention, including but not limited to, a conventional computer keyboard, conventional computer mouse, infrared remote control device, RF remote control device, joystick, handheld computer, etc. The input devices in the preferred embodiment have been found to offer better functionality and ergonomics in the typical examination room. This hardware is combined with custom software, which accurately presents tests, charts and optotypes to the precise specifications of each healthcare professional.

A process for testing the visual acuity of the patient's eyesight is also provided. The system was designed to allow nearly every input, screen layout, screen content, screen output, calibration and behavior to be customized to physician's_specifications or requirements based upon the examination room environment and the physician's preferred method for conducting an exam. For example, the system allows each ophthalmologist to specify the acuity tests, letters, pictures and optotypes used in their eye examinations. The process includes initiating a VAT from a CPU using a controller. The VAT is then displayed on a display device that is operatively associated with the CPU. Furthermore, a controller is used to initiate eye fixation images used in conducting eye assessments to be displayed on the display device. The VAT and the eye assessment data is presented on the CPU using visual acuity and fixation control software that has been previously installed onto the CPU.

In a preferred embodiment, the process of testing the visual acuity of the patient's eyesight includes an integrated contrast sensitivity test. The contrast sensitivity test incorporated has been enhanced to test infinitely variable contrast levels and infinitely variable colors, backgrounds, characters or optotypes.

Still other advantages of the present invention will be recognized upon a reading and understanding of this specification by one of ordinary skill in the art.

### DESCRIPTION OF THE DRAWINGS

The present invention may take form in various components and arrangements of components, and/or in various steps and arrangements of steps. The drawings are only for purposes of illustrating preferred embodiments and are not to be construed as limiting the invention.

FIG. 1 illustrates a preferred embodiment of the visual acuity and fixation control system, including a CPU, a monitor with an audio output, a keypad controller, a foot pedal controller and software.

FIG. 2 illustrates a preferred embodiment for a three-part foot pedal controller used for operation of fixation targets according to the present invention.

FIG. 2A is an elevational view of the controller of FIG. 2.

FIG. 3 illustrates one embodiment for a keypad controller for control of VAT according to the present invention, the LCD backlit display and phosphorescent keys are also indicated.

FIG. 4 is a flow diagram illustrating one embodiment for a routine for initiating eye fixation images for eye assessments.

FIG. 4A is a flow diagram illustrating a routine for adding and deleting fixation targets.

FIG. 5 is a flow diagram illustrating a preferred embodiment for a routine for requesting initiation of a VAT.

FIG. 6A shows a single line of letters and FIGS. 6B and 6C show use of an optotype pointer.

FIGS. 7A-7D are representative screens demonstrating the contrast sensitivity test.

FIG. 8 is a flow diagram illustrating a preferred embodiment for termination of a fixation display for an eye assessment or instantaneously switch to a VAT.

FIG. 9 is a flow diagram illustrating one embodiment for a routine for requesting an acuity test and level to turn off or instantaneously switch to a fixation target.

FIG. 10 is a flow diagram illustrating a preferred embodiment for requesting all acuity tests to be properly calibrated for the size of the examination room and for examination rooms which utilize mirrors for acuity test display.

FIGS. 11A-11F are representative screens illustrating how the VAT and VACFS can be customized and calibrated.

FIG. 12 is a flow diagram illustrating a preferred embodiment for a routine for converting each acuity test basis for acuity measurement to the Logmar measurement standard.

### DETAILED DESCRIPTION OF THE PREFERRED EMBODIMENTS

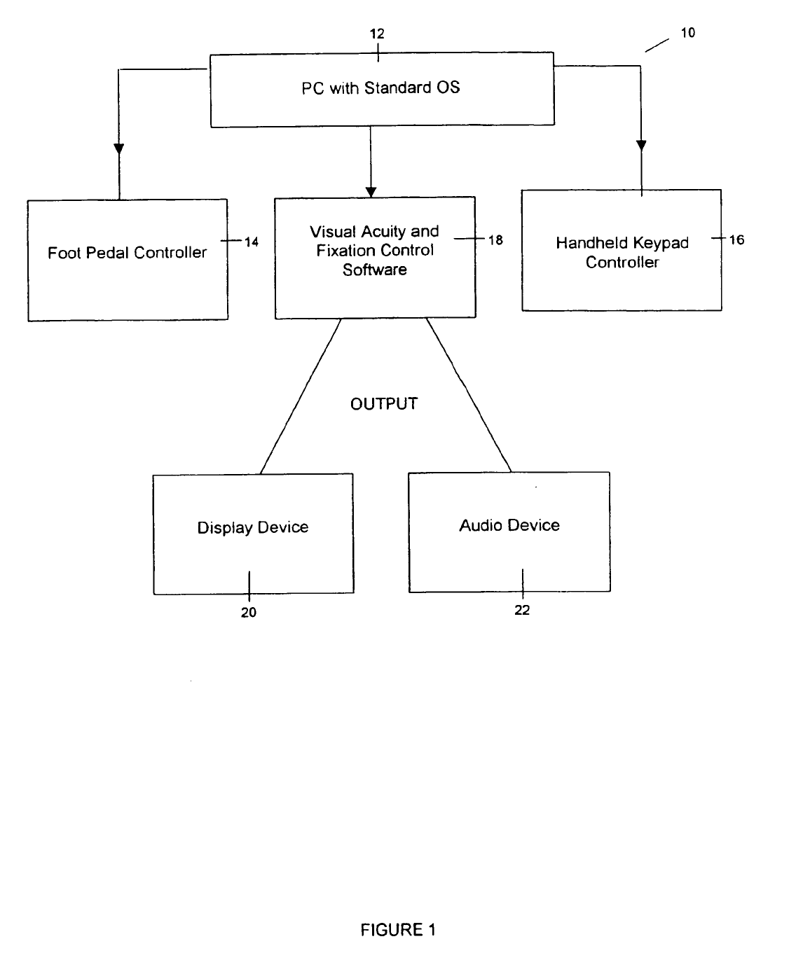

In accordance with one aspect of the present invention, an apparatus and method for conducting a visual acuity test (VAT) is provided. FIG. 1 schematically illustrates a computer-based system in accordance with a preferred embodiment of the present invention. The system includes a conventional computer 12 or central processing unit (CPU) with an operating system, such as Windows®. It is contemplated, however, that the present invention can be designed to be compatible with other computer operating systems, such as Apple®, Linux®, UNIX and so forth. The apparatus comprises a display device 20 and audio device 22 used in conjunction with a computer 12, a first controller or foot pedal controller 14, a second controller or handheld keypad controller 16 used in conjunction with control software 18 to provide video and audio output to a display device 20 and audio device 22, respectively. The method according to the present invention includes the use of a particular arrangement of components for conducting a VAT. Alternatively, it is also contemplated that the display device can be integrated into one unit with a CPU, for example, in using a laptop to conduct the VAT.

Preferably, customizable data, images and audio are displayed to a patient using a conventional personal computer including the display monitor 20 and the audio device 22 operatively associated therewith in order to conduct a VAT. It is contemplated, however, that the present invention may be relevant to practice in other applications throughout the healthcare field, and as such, the following description of the present invention should not be construed as limiting the present invention in any way.

The foot pedal controller 14 and handheld keypad controller 16 are used in conjunction with the control software, also referred to as Visual Acuity and Fixation Control Software (VAFCS) 18 which enables a health care professional in an examination room setting to display customized data, images and audio for use in conducting VATs and for fixating an eye of a patient in the course of examining the eye for disease, disorder and/or astigmatism.

VAFCS 18 allows each health care professional to individually configure their system tests. This is accomplished by establishing a substantial configuration file that contains parameters that define both the behavior and the appearance of the system. Parameters that relate to the health care professional's set-up and configuration are maintained by using the system configuration screens that are available within the control software 18. The configuration file or list exists that defines every button, key and switch that can potentially generate an input that will be intercepted by the VAFCS. Preferably, during initial set-up of the VAT system, the health care professional specifies the program function required on each button of the keypad of the second controller, and each switch of the first controller. These assignments can then be entered into a configuration file during set-up of the system. In addition, the health care professional may re-assign any key, button or switch either from within the software configuration screen or by altering the codes associated with each of the inputs listed in the configuration file that need to be changed based on the preferences of the health care professional. The health care professional may elect to assign the code associated with any test, mode or function to more than one key, button or switch (for example, the health care professional may elect to assign “Blank Screen” function to both a key on the keypad and a switch on the foot pedal). Then, in operational mode, when the user depresses a key, button or switch to obtain a test, mode or function, VAFCS looks up the test, mode or function in the configuration file or list and initiates the test along with any additional subprogram required by that item in the configuration file. Thus, by having assigned keys or buttons within the system the control software 18 and the first and second controllers allow for seamless integration into and between video fixation, VAT character generation and animated targets.

The foot pedal controller is preferably the control device for all eye assessments where eye fixation is required. The health care professional depresses one section (of any desired number of sections) of the foot control pedal to display the desired eye fixation target. Preferably, there are three sections of the foot pedal controller, each initiating a different eye fixation target on the display device. Each section within the pedal unit is assigned pre-determined fixation functions, which can be assigned by the health care professional.

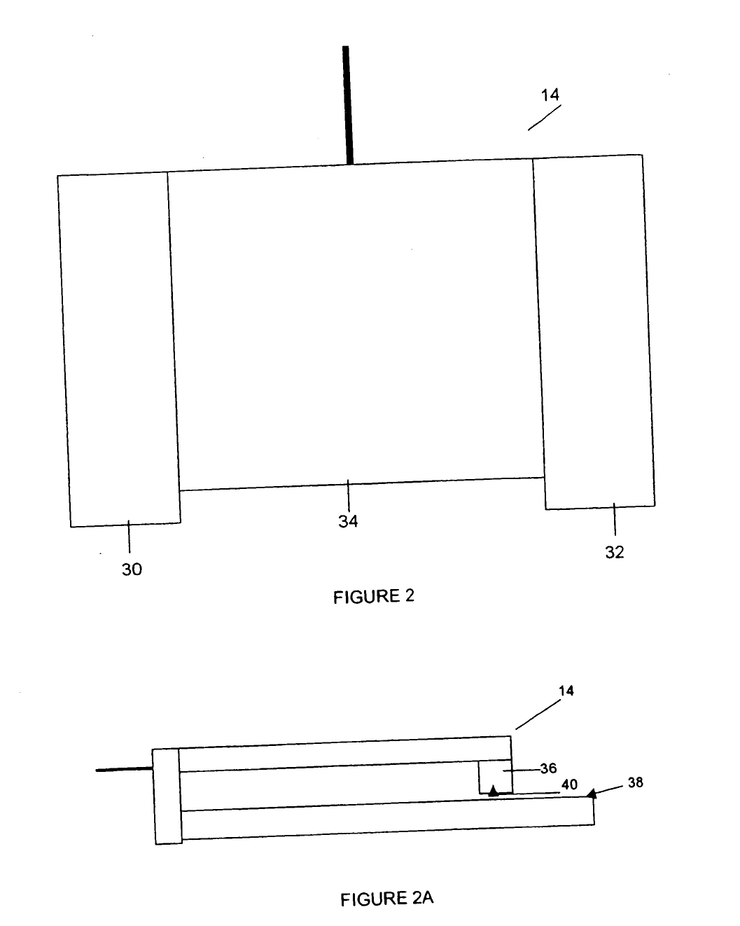

As shown in FIG. 2, a preferred foot pedal controller 14 according to the present invention has three pedals 30, 32, 34, any of which are used to control animated fixation targets 30, 32, or video movies 34. The foot pedal controller 14 is typically located within a convenient distance, i.e., within arm's length, from the health care professional for easy access and control. The health care professional can purposely control his/her foot movement to obtain the required fixation target to expedite the examination process as well as assist in the diagnosis of certain eye disorders. The unit is preferably made from a high impact plastic designed to withstand thousands of foot contacts to ensure long term reliability.

The foot pedal controller includes a triggering system, not unlike that of a computer mouse, which is capable of sending signals from each individual section of the foot pedal controller. The contact information within each section of the foot pedal controller is processed and converted into a digital signal, which is then sent to a digital I/O port and relayed to the CPU. Depending on the signal sent the result is to “turn on” or “turn off” a specific animated target or video movie.

FIG. 2A illustrates a preferred embodiment of a switch 36 according to the present invention, where each section of the foot pedal controller 14 includes a separate switch for each section. Each switch is preferably a single-pole-single-throw-type switch, each operating independent of each other. The internal switch of each pedal is a two-piece staggered copper strip 38, 40 which makes instant contact upon depression of the pedal. Contact is made between the lower section 38 of the pedal and the contact 40, which is attached to the upper section of the pedal. Once contact is made, a digital signal is sent and a specific video and audio action occurs as a result of the CPU receiving such a signal from the foot pedal controller. This action will continue until any other pedal section on the foot pedal controller or key on the handheld keypad is depressed. Of course, it will be appreciated that this switch is preferred, although alternative switches that achieve the same functions and advantages offered by the present arrangement can be used without departing from the scope and intent of the present invention. The cabling connector from the foot controller to the digital I/O port can be a USB, PS/2 type plug, a serial connection, a parallel connection, or any other suitable connection.

The health care professional has complete control of the length of time that each fixation target and/or VAT is displayed on the display device by using the handheld keypad and/or the foot control pedal, respectively. The display can be as short as a few seconds or can seamlessly replay a video/audio display as many times as necessary for the health care professional to rule out certain eye disorders.

The foot control pedal is the preferred control device for all eye fixation tests, and is operated by the health care professional by depressing any one of a number, preferably three, sections of the foot control pedal to display the desired eye fixation target.

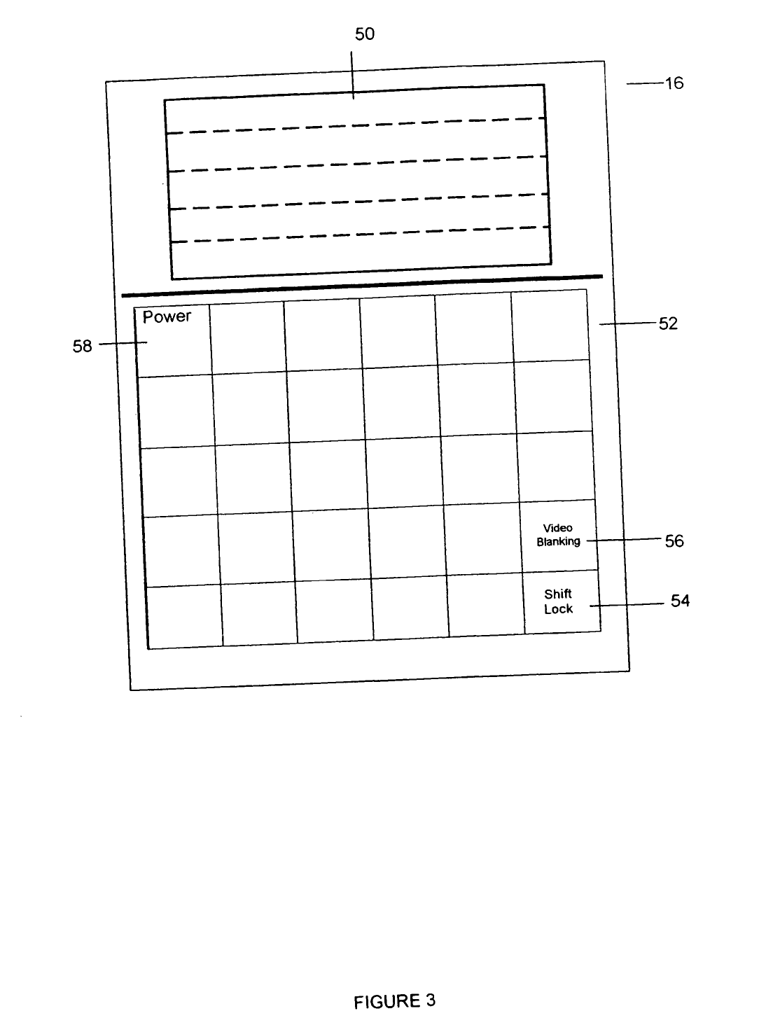

FIG. 3 illustrates one embodiment for a handheld keypad controller 16 in accordance with the present invention. The keypad controller is the device that controls which VATs are administered, although it is contemplated that the VACFS can be manually run from the personal computer as well. The keypad controller relays information concerning each individual VAT to the health care professional via an LCD 50 located on the top surface of the unit. As the origination point of clinical testing, the keypad controller interprets an individual scan code when a key from the keypad 52 is depressed and then sends the required information to both the display device associated with the personal computer and the LCD associated with the keypad controller. As shown, the keypad unit 52 is an electromechanical device disposed adjacent the backlit LCD 50 on the keypad controller 16. A connection is made to a serial digital I/O port and the unit is preferably powered by a portable power source, such as a traditional battery, like a 9-volt DC source. The keypad controller is located in the examination room within the immediate proximity of the health care professional.

The keypad controller preferably includes a liquid crystal display (LCD) that is connected to a digital I/O port. The LCD is preferably backlit to allow the health care professional to operate the keypad controller within the examination room in the event that the lights in the room are dimmed or off, which is sometimes done in order reduce distraction or to provide desired contrast between the display device and the ambient light within the room. This allows the patient to see the display device more clearly. The keypad controller has, for example, a performance capability of accessing approximately fifty independent tests utilizing contact square surface area keys. The keypad is preferably handheld, however, it is contemplated to alternatively be used as a tabletop controller, such as on a refraction counter. The top surface of the keypad is preferably sloped from the back of the unit to the front of the unit in order to provide an easier view when holding the unit parallel to the ground. Preferably, the top surfaces of the individual keys in the keypad controller are capped with a blank phosphorescent material for display in low or no light conditions. These keys remain bright through the use of light emitting diodes that recharge the phosphorescent keys throughout the day. Key text is added for the individual acuity tests and levels of tests after each health care professional selects the custom charts, pictures, and specific optotypes required in their examinations.

As shown, the handheld keypad controller has preferably about thirty individual keys, although it is contemplated that any number of keys can be included in the keypad. The keys within the keypad 52 are used to display the following types of tests: standard eye charts, pictures, tumbling “E”'s, crowding bars, numbers, single characters, HOTV letters, red/green block out, contrast sensitivity test and astigmatism measurements. In addition, some of the keys on the keypad could be used for other purposes, such as a shift button 54, a video blanking button 56 and a power button 58. The shift button 56 enables the health care professional to instantaneously switch to alternative visual acuity tests, fixation targets or functions. The power button 58 can be programmed to only turn off the keypad controller unit. Of course it should be appreciated that the keypad controller is preferred, although alternative controllers that achieve the same functions and advantages offered by the present arrangement can be used without departing from the scope and intent of the present invention. For example, it is contemplated that a personal digital assistant (“PDA”) can be adapted for use in the present invention as a keypad controller to be held by the health care professional in initiating the VAFCS program.

The VAFCS is a computer program that utilizes eye charts, pictures, tumbling “E”'s, crowding bars, numbers, single characters, HOTV letters, red/green blockout, contrast sensitivity tests, astigmatism measurements and video fixation. The calibration of the components is based on the distance from the display device to the patient. The input of this measurement is required only once in the initial setup and can accommodate a mirror based examination room, however, the system can be recalibrated as explained below.

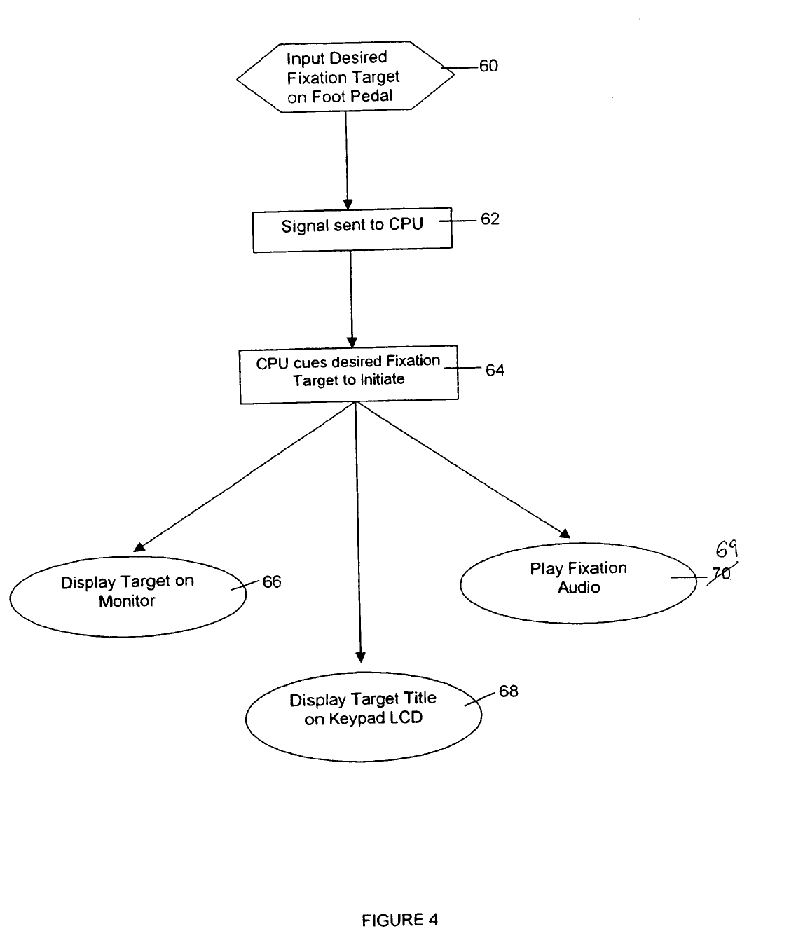

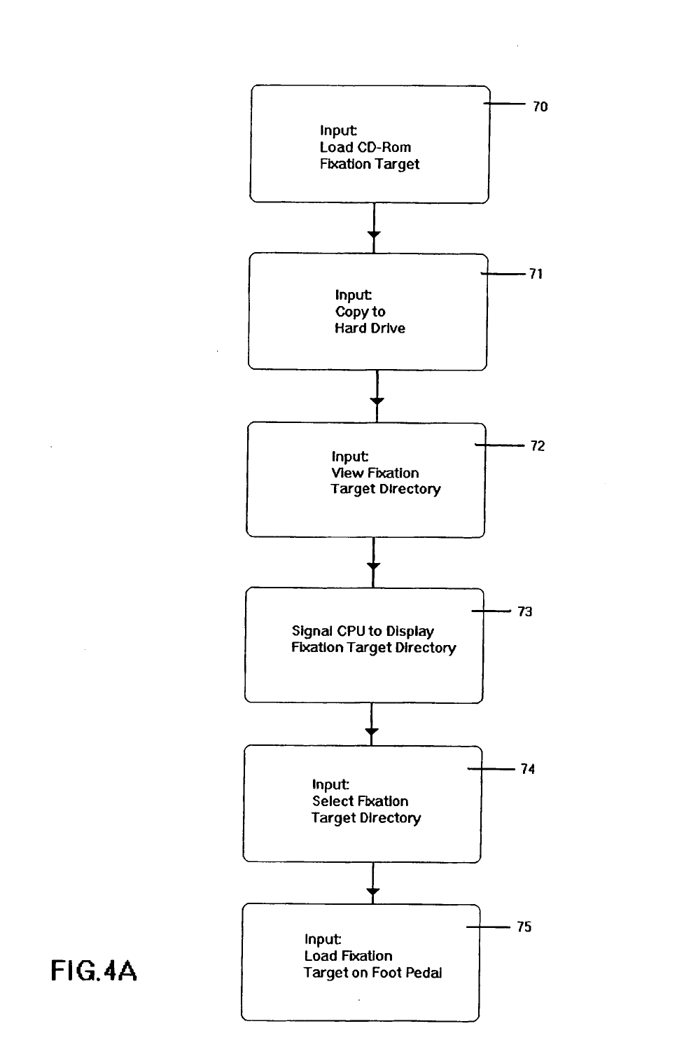

FIG. 4 illustrates a preferred embodiment for a routine that initiates the eye fixation images for eye assessments. The action is initiated by the health care professional depressing one of the three sections on the custom foot pedal controller as represented in step 60. Each section controls one of the three health care professional designed image targets or videos. Upon depressing the foot pedal, a signal is sent to the CPU in step 62 and the VAFCS translates the signal from the foot pedal into the desired pre-determined graphic (PDG) in step 64. The PDG is relayed to the CPU, which then communicates this to the monitor, keypad LCD and audio speakers. The monitor will then display the PDG as per the health care professional's design as represented in step 66. The audio speakers will play in step 69 in coordination with the visual display on the display device and the keypad LCD will define, in text, which fixation target and/or VAT is currently being initiated as indicated in 68. The VAFCS serves as the controlling mechanism for the visual and audio display on the monitor and speakers by recycling through the fixation segment periodically.

FIG. 4A illustrates a routine that enables a physician to add and delete movies and fixation targets on the CPU hard drive 12 and then update this onto the custom foot pedal controller 14. The action is initiated by the physician loading a new movie or fixation target as represented by step 70 into the hard drive via a CD-Rom (1A). The physician copies the fixation target onto the CPU hard drive as noted in step 71. Upon completion, the physician can depress an assigned key on the custom keypad controller (see step 72), which signals VAFCS to search the hard drive for all movies and fixation targets. Once found, VAFCS displays the fixation target on the monitor (4J) (step 73). The physician then scrolls the list using keys on the keypad. Once the desired movie is highlighted, the physician can depress the key 74 on the custom keypad controller, which will locate the desired movie or fixation target on pedal 1, 2 or 3 on the custom foot pedal controller. Once depressed, a signal is sent to VAFCS, which runs a routine 75 to replace the original movie or fixation target with the newly selected one.

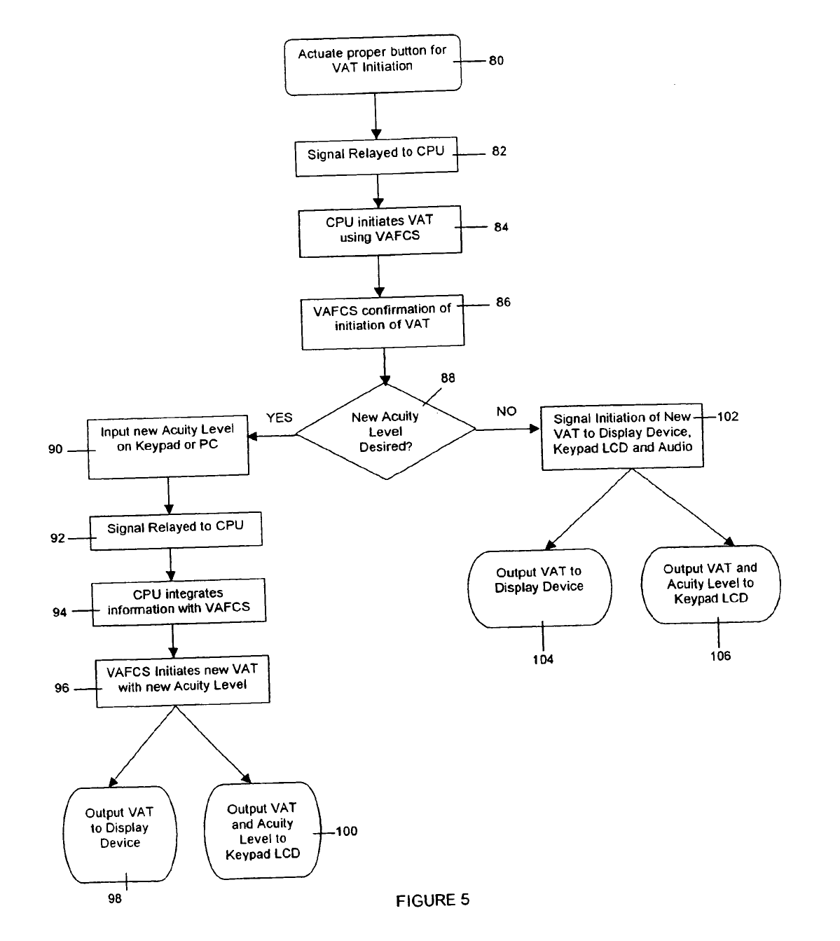

FIG. 5 illustrates one embodiment of a routine that initiates a VAT in accordance with the present invention. The VAT is initiated by the health care professional depressing one of the healthcare professional's specified acuity type test keys on the custom keypad controller in step 80. Each key is preferably dedicated to one or more of the following acuity tests: Snellen/Logmar, pictures, tumbling E's, crowding bars, numbers, single characters, HOTV, red/green, contrast sensitivity and astigmatism (all are customary acuity tests, although the list should not be restrictive since newly developed tests may also be accommodated). Upon actuation (i.e., pressing) of a key, the signal is sent to the CPU in step 82 and the VAFCS translates the signal from the keypad into the desired VAT and/or video fixation in step 84. The health care professional decides whether another acuity test level is required. If no new acuity level is desired, the VAFCS will instruct 102 the keypad LCD to display the specific VAT as referenced at 106 and test level being presented on the display device as referenced at 104. The VAFCS is normally programmed to remember the last acuity test level that was presented and can incorporate both the test and level into its instructions to the CPU. The desired VAT is displayed on the display device as per the design of the health care professional at the acuity level last presented in an acuity test. If a new acuity test level is desired, the health care professional can select any available acuity test levels (for example; 20/20 is a separate key from 20/40) as noted in step 90. The signal to change the acuity level is relayed to the CPU in step 92, which integrates that information with VAFCS in step 94 to initiate a new VAT with a new acuity level in step 96. The VAFCS also instructs the keypad LCD to display the specific VAT as referenced at 100 and test level being presented on the display device as referenced at 98.

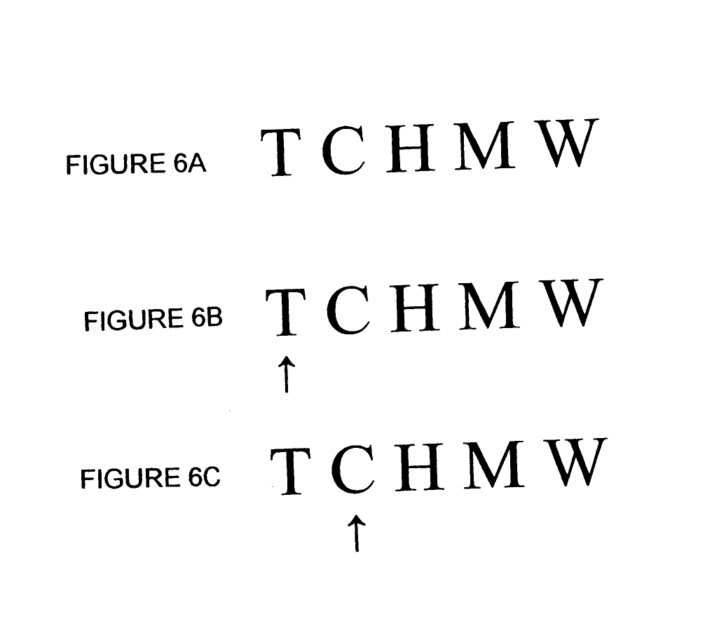

In addition, once the VAT has been initialized it is possible for the health care professional to present the VAT in single lines of letters, pictures or optotypes. In the preferred embodiment, the actions are initiated by the health care professional depressing a key on the keypad controller. The VAFCS then translates the signal from the keypad and will adjust the VAT to present only a single line of letters, pictures, or optotypes. The single line of letters, pictures, or optotypes that will be displayed are those that were previously selected by the health care professional during the initial set up of the system. FIG. 6A shows a single line of horizontal optotypes. Once the single line of characters is selected, it is also an option of the health care professional to display an optotype pointer (FIG. 6B) that can be shown beneath or beside any single line of a vertical or horizontal acuity test. The action can be initiated by depressing a specific button on the handheld keypad after having selected a single line test. The VAFCS then translates the signal received from the handheld keypad controller and retrieves the arrow optotype from the loaded data within the software. Typically, once initiated, the VAFCS will cause the optotype pointer to appear under the furthest left hand side or beside the top letter, picture, or optotype to be displayed on the monitor. Then, by depressing an assigned key on the handheld keypad controller, the optotype pointer will move from left to right beneath the characters in a single horizontal single line (FIG. 6C) or from the top to bottom in the vertical single line being tested. By depressing the key that caused the optotype line to appear a second time, the optotype arrow will disappear from the screen and a single line of letters, pictures, or optotypes will remain displayed on the monitor with no arrow or pointer underneath the characters.

Alternatively, it is possible to cycle a single letter, picture, or optotype VAT on the monitor. In this embodiment, a single letter, picture, or optotype is displayed on the monitor and by depressing a designated button on the handheld keypad, the VAT character designations that go along with the VAT are displayed one at a time and cycled through as opposed to being displayed all at once. The cycling feature can be activated by again selecting a VAT to be used and then depressing a key on the handheld keypad that is assigned to the cycling single optotype display. Once the cycling button on the handheld keypad is depressed, the letters, pictures, or optotypes are displayed one at a time for a designated period of time and then cycled through automatically without the health care professional having to depress the key to move to the next letter, picture, or optotype. The single line of letters, pictures, or optotypes will cycle through automatically until the health care professional depresses the cycling button again on the handheld keypad controller which will cause the cycling action to cease and yet still display the single letter, picture, or optotype that is on the monitor at the time of termination of the cycling feature.

It is also contemplated that a test mode can be used in conjunction with a selected acuity test. The action is initiated by the health care professional i depressing one of the test mode keys on the keypad controller. Upon depressing one of the keys, the control system 18 translates the signal from the keypad controller into a display of the test mode to be overlaid upon the selected acuity test. For example, if a health care professional is displaying Allen pictures, by pressing the button assigned to a crowding bars, then the Allen pictures are presented surrounded by crowding bars. The control software, or VAFCS 18 instructs both the keypad controller LCD and the display monitor to display the desired test and mode. The test mode continues until a different mode or test key is depressed by the health care professional which VAFCS interprets as a resuming of standard testing without a mode overlay. The VAFCS operates using a number of internal program modes that retain a value at all times. For example, if the size is set to 20/40, the displayed size will remain 20/40 for all test modes and submodes until the size is deliberately changed. Similarly, a high level “test mode” will remain unchanged, with a few exceptions, until deliberately changed. If the key to initiate Snellen charts as the selected test is depressed, the high level test mode is Snellen and will remain in effect for the standard chart, single line, single line with arrow, single character, cycling single character, 50% or 100% crowding bars, red/green blockout, vertical line and the same size lines. The mode will remain in Snellen while any size optotype is selected and the display will return to Snellen when any fixation target, picture or “.jpg” is selected and then turned off. This high-level test mode will be turned off only when a new test mode is selected.

Additionally, this system preferably can handle contrast sensitivity as a test mode. The action is initiated by the health care professional depressing the keys assigned to contrast sensitivity test mode on the keypad controller or the foot pedal controller, or some other input device. Upon initiation, the VAFCS 18 translates the signal from the keypad controller into a display of the contrast sensitivity test mode to be overlaid upon the selected acuity test. For example, if a health care professional is displaying Allen pictures, by pressing the contrast sensitivity key, the Allen pictures are presented in varying levels of color muting or grayed density, which can be adjusted in increments ranging from 0% to 100% using the keys assigned preferably with “+” or “−”. It is also contemplated that by using a separate key the background color or density can be adjusted in increments ranging from 0% to 100% in the same manner as the image or optotype was adjusted. This can be used for conducting an alternate version of low contrast testing. The contrast sensitivity testing uses images displayed on the display monitor that have the output intensity of the image set to a known percentage that represents a reduction in the brightness from a minimum of 1% to a maximum of 100%. These images are displayed on a background that is set to a known percentage that represents a reduction in the brightness from a minimum of 0% to a maximum of 100%. The contrast between the background and the images displayed on the background is then measured as the difference between the brightness of the character or image displayed as a percentage reduction from its maximum and the brightness of the background displayed as a percentage reduction from its maximum. This offers the advantage of displaying an image intensity of a known percentage of brightness to a known intensity of the background.

This method eliminates errors inherent in the use of an uncontrolled reflected or projected light source and an image that is physically printed on a surface, as well as errors in computer projections based on the current settings of the monitor. Therefore, for both the images presented and the background, the “whitest white”, for example, the monitor is capable of at its current state of adjustment is represented as 0% reduction in pixel intensity from maximum “white”. Furthermore, the “blackest black”, for example, that the monitor is capable of at its current state of adjustment is represented as a 100% reduction in pixel intensity from maximum “white”. Since the percentage reduction of intensity from “maximum white”, for example, is controlled as a continuous linear reduction of pixel intensity on the monitor, the absolute luminance of maximum white can be controlled by the user by adjusting the monitor, but the intensity of pixel luminance relative to “maximum white”, for example, can be accurately controlled by the software.

The density of the image is described as a “pixel density” relative to the maximum white that the monitor is capable of producing in its current state of adjustment. The density of the background is described as a “pixel density” relative to the maximum white that the monitor is capable of producing. For example, an image of 1% “pixel density” is an image where the pixels that make up the optotype are illuminated at 99% of the intensity of the monitor's maximum white. If the background is set to white, a 0% reduction in pixel density, displays the optotype at 1% contrast. On the other hand, if the background is set to a 3% reduction in pixel intensity from the monitor's maximum white, then the contrast of the same optotype is a −2% contrast. Additionally, since the color dots (red, green, blue) that compose a pixel on a standard computer monitor can be independently controlled, the system allows an overlay of a nearly infinite range of color on the pixels that compose both the optotypes displayed as well as the background. Therefore, the system allows contrast sensitivity to be measured using a range of pixel density from 0% (white) with any selected color being injected in a continuous range from greater than 0% to 100% until, at 100%, light output is suppressed entirely (i.e., black). In the range of pixel intensity ranging from greater than 0% reduction in intensity to less than 100% reduction in intensity, the optotype is displayed in the user-selected color. For example, by selecting a hue of green for the background and a hue of yellow for the optotypes, it is possible to measure the patient's ability to discern the optotype as intensity and background intensity are altered. As with the same process conducted with the colors set to neutral (white/gray/black), the contrast can be represented as the difference between the brightness of the character displayed as a percentage reduction from maximum intensity and the brightness of the background displayed as a percentage reduction from maximum intensity. As an example, if a blue background is set to a 30% reduction in intensity from maximum and a green optotype is displayed at a +28% reduction in intensity, the resulting contrast can be expressed as −2%. On the other hand, if the optotype is set to a 43% reduction in pixel intensity from the monitor's maximum, on the same background, the contrast of the same optotype relative to the same background is a 13% contrast.

The system is composed of multiple sets of images ranging in pixel density and optotypes of a pixel density that can be varied from 0% to 100% and a background that ranges in pixel density from 0% reduction in pixel density (e.g., white) to 100% reduction in pixel density (e.g., black) in white/gray/black or any color. The health care professional can select the test mode (i.e., Snellen, numbers, Lea Symbols, etc.) used in normal acuity testing and then press a key that is assigned to contrast sensitivity. The selection of the key assigned to contrast sensitivity causes the selected optotype to be displayed at a predetermined contrast (the difference in intensity between the optotype and the background) of which the optotype is preferably a shade of gray, but can be any color, and the background is preferably white or black, but can also be of any color and intensity. The health care professional can then increase or decrease the pixel density or colors of the optotypes or background by depressing keys on the keypad controller.

The system can also be customized for the specific contrast sensitivity test conducted by each health care professional. Specifically, both the background and the optotypes can be designed to resemble any character, image or appearance used in testing. For example, if required, a health care professional that examines pilots could display a custom background that resembles night in adverse weather conditions with optotypes that resemble the images one would see on a runway.

The contrast sensitivity test provides a method of testing for the point at which the test images disappear from the patient's vision. Similarly, the health care professional may choose to begin testing at a contrast level the patient cannot see and increase the pixel density to determine the contrast level where the optotypes become visible to the patient. For this reason, the optotypes can be accurately varied in contrast in 1% increments from an image that is only very faintly visible on close inspection by a person with normal vision to a relatively dark gray that is visible to most patients.

The contrast sensitivity test is inherently repeatable and consistent due to the fact that the pixel illumination intensity is precisely controlled in 1% increments relative to the pixel illumination of the background. Furthermore, due to the ability to customize the color spectrum and the actual optotypes and background appearance, the system offers the advantage of being able to test contrast sensitivity in any environment that a patient interacts, as well as with any standard visual acuity test screen. For example, during the course of a normal visual acuity examination, the health care professional will determine the acuity level of the patient. Then, if desired, the health care professional can overlay the contrast sensitivity test mode over any of the acuity tests, thereby enabling simultaneous testing of low contrast on any test chart for the patient's acuity level.

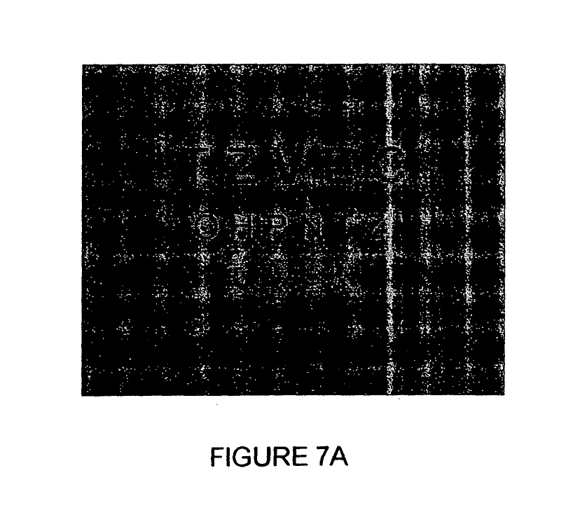

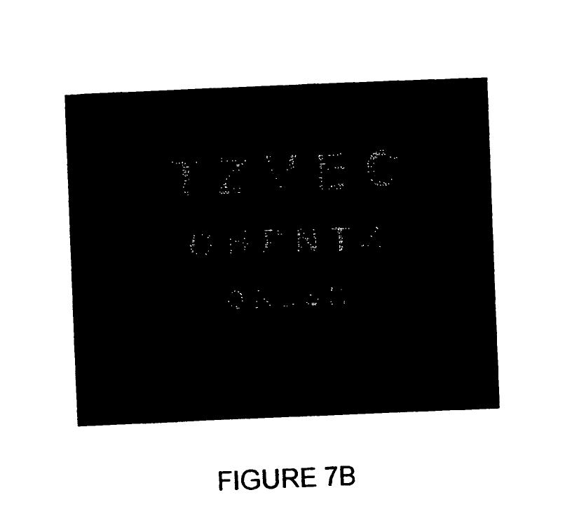

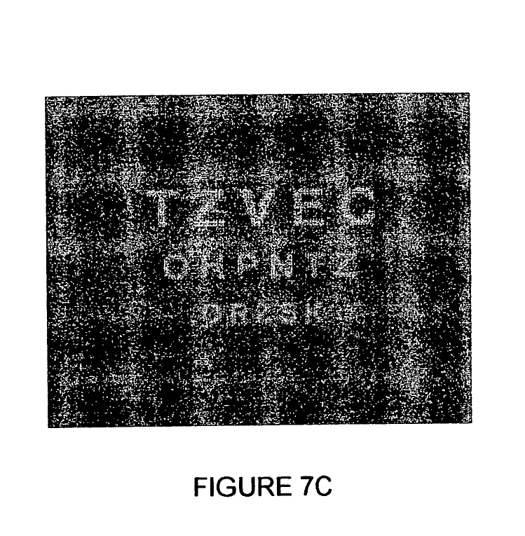

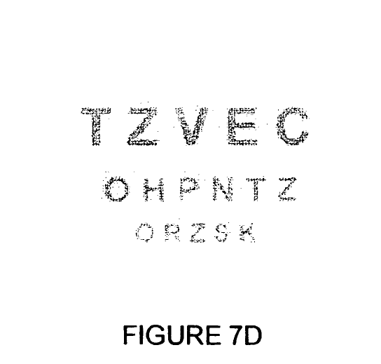

FIGS. 7A-7D illustrate representative screens demonstrating the contrast sensitivity test capabilities described above. As is apparent, the background and/or the optotype can be varied through a range of pixel densities to provide a wide array of contrast sensitivity heretofore incapable of accurate assessment. Although FIGS. 6A-6D are shown in gray scale, the use of different colors for the background and/or optotypes, in conjunction with the ability to provide controlled images, pixel densities, etc., provide the health care professional a tool and test capabilities that were previously unavailable.

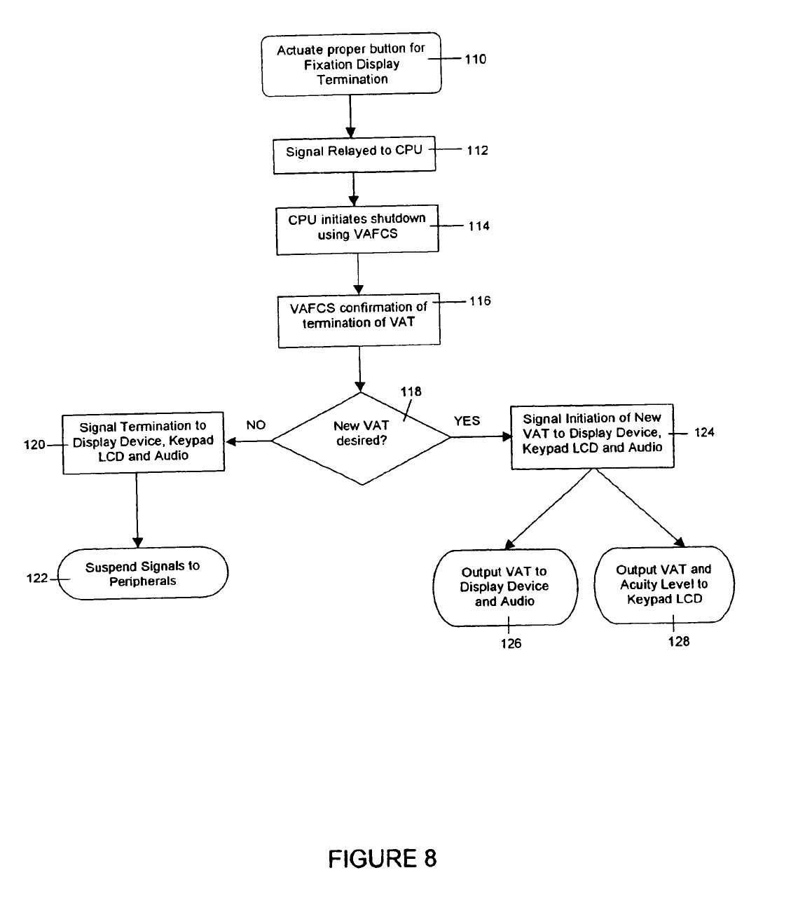

FIG. 8 illustrates a preferred routine for terminating the eye assessment which uses the fixation image. The action is initiated by the health care professional either depressing one of the three sections on the custom foot pedal controller or by depressing a key on the custom keypad controller as identified in step 110. Upon depressing the foot pedal or key on the keypad, a signal is sent to the CPU in step 112. The VAFCS translates the signal to initiate shutdown in step 114 and confirms whether the output devices should be fully terminated at step 116 or whether another fixation target or acuity test at decision step 118. VAFCS will either provide instructions to the CPU that will darken the monitor and keypad LCD and mute the audio speakers at step 120 to suspend signals to the peripherals as represented at 122 or will cause the next desired fixation target or acuity test to appear at step 124 on the display device and audio speaker as represented at 126 to the keypad LCD as represented at 128.

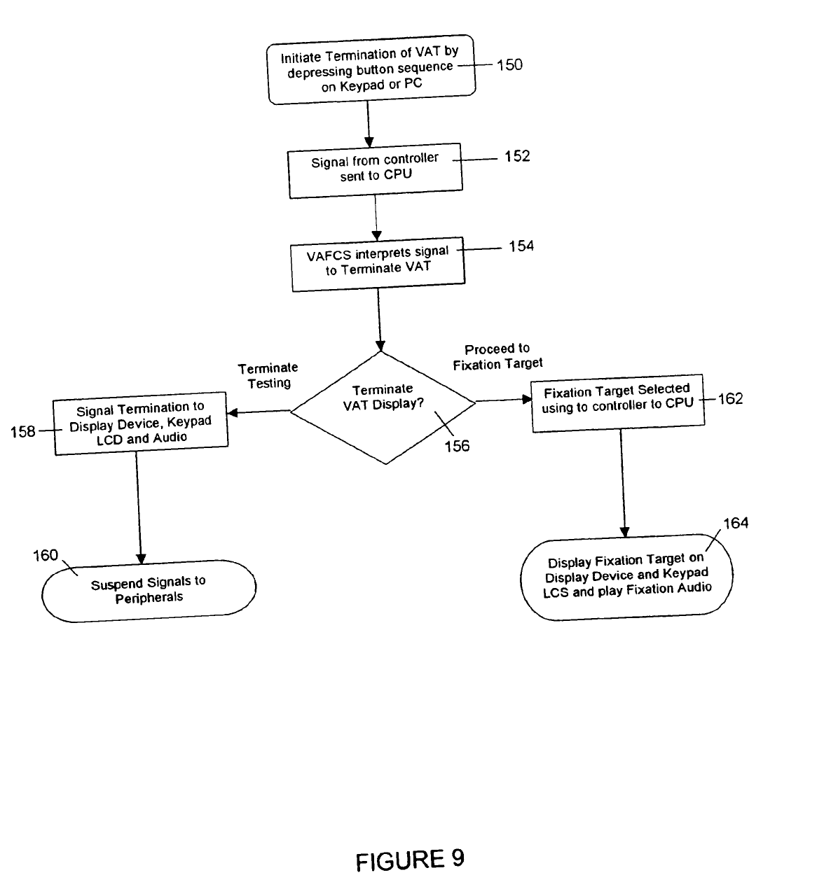

FIG. 9 illustrates one example of a typical shutdown routine for the VAT system in accordance with the present invention. The shutdown of the system is initiated by the health care professional depressing a single key on the custom keypad controller, by depressing one of the three sections on the custom foot pedal controller, or by manually shutting down the system at the personal computer 150. Upon depressing the designated “off key” on the keypad or a particular section of the foot pedal controller, a signal is sent to the CPU as indicated in step 152 and the VAFCS translates the signal in step 154 into whether the output devices should be totally turned off or whether another acuity test or fixation target is desired at decision step 156. VAFCS will either provide instructions to the CPU that will darken the monitor and keypad LCD and shut off audio at step 158, thereby suspending signals to peripherals as represented at 160, or will cause the next desired acuity test or fixation target to initiate as represented at 162 on the display device, keypad LCD and audio speakers as indicated at 164.

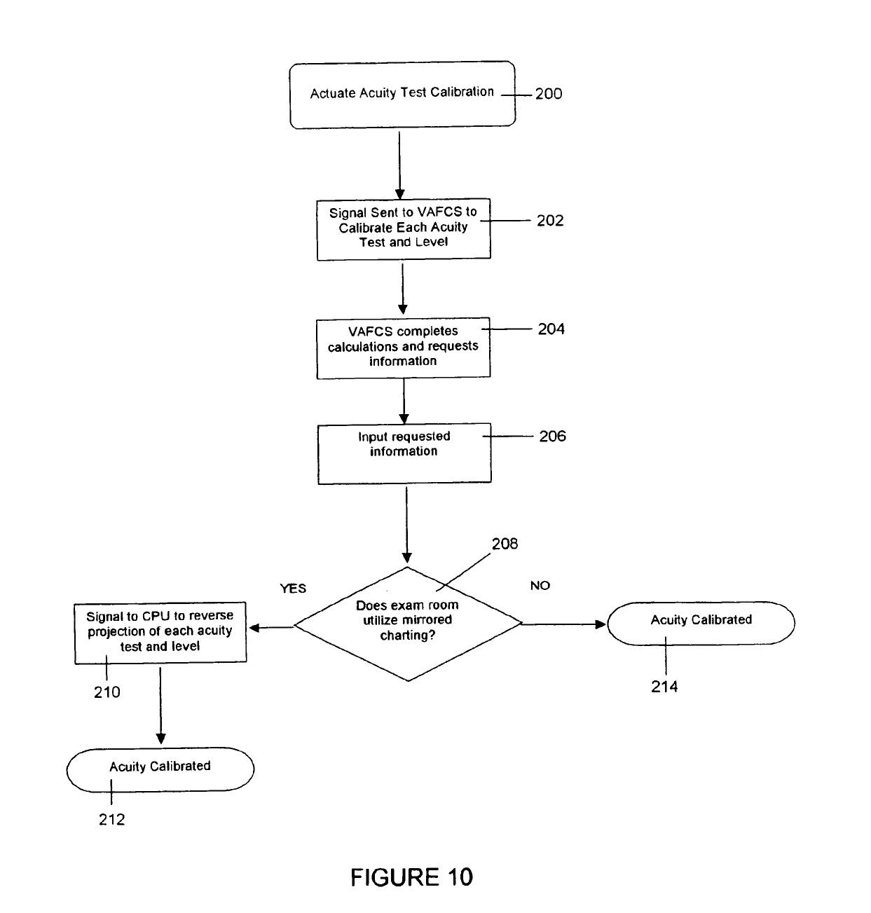

FIG. 10 shows a preferred embodiment of how each of the acuity tests is calibrated to the size of the room and whether a mirror is used as the focus point for visual displays. The calibration action is initiated by the health care professional initiating such action on the VAFCS, typically by utilizing a conventional keyboard to input as represented in step 200. Using the keys, the health care professional is able to input the examination room size. Once input, VAFCS is signaled to calibrate each acuity test and level in step 202. The VAFCS will take this input and begin calculations on each of the acuity tests and levels to ensure that each test and measurement precisely displays the size of the test, picture or specific optotype in accordance with the desired acuity level at the time of the examination in step 204. VAFCS can store the calibration information and utilize this information every time it instructs the CPU which VAT to initialize. In addition, VAFCS can request more information regarding the preferences for each acuity test and level 206, such as whether the health care professional uses mirrors as the primary display medium in an eye examination. The health care professional can utilize the keyboard to input whether a mirror is in use in the exam room as noted in step 208. If a mirror is used, VAFCS will take this input and begin reversing each acuity test into its mirror image in 210. VAFCS can store the mirror image information and utilize such information every time it instructs the CPU which VAT to initialize. By using the reversed image on the monitor, the mirror will always display the text, picture or specific optotype in the correct, desired configuration, thus calibrating the acuity test as indicated in step 212. If no mirror is used, then once information regarding testing preferences is submitted to VAFCS, and that information is integrated, the acuity test is calibrated 214.

Based on the room length entered in by the health care professional as stated above, VAFCS precisely calculates in twips (a standard unit of graphic size that is independent of resolution on a monitor) and the height and width of the characters. During the initial set up process, one of several resolution options is preferably automatically selected through VAFCS ensuring that the appearance of each letter or image is the best for each size. Several image files of varying resolution level are included with the VAFCS and are stored within the hard drive of the CPU. Using the length of the room and the size of the optotype in twips, the image file, which is stored on the hard drive, can then be increased in size or decreased in size with the clearest image being automatically selected by VAFCS. VAFCS will then perform a validation of the image selection by proofing the mathematics of the required image adjustment from the selected image file. If an alternate image file, however, interpolates better mathematically, the selected image file will be revised. Importantly, VAFCS uses interpolation and calculation for sizing the images which is used in combination with the automatic resolution selection at set up. VAFCS will permanently store the calibration and utilizes this information every time an acuity test is initiated.

In addition to being able to calibrate an acuity test depending on the size of the room and whether a mirror is used, different parameters can be set during the initial set up of the system. Different acuity tests, sizes, fixation targets and the selected letters or pictures with each test, the line configurations including randomization and cycle time for automated letter or picture cycling are examples of the types of parameters that can be set up initially by the health care professional. The parameters can be entered into the VAFCS during the initial set up using a conventional keyboard. The parameters are then interpreted by VAFCS and stored on the hard drive of the CPU. Following the parameter selection by the health care professional, VAFCS will interpret the critical size parameters (e.g. room length for testing) and selects an optimal image resolution size for each letter, picture and optotype that is stored as a preference and is used each time the particular acuity test to which the parameter is correlated is initiated.

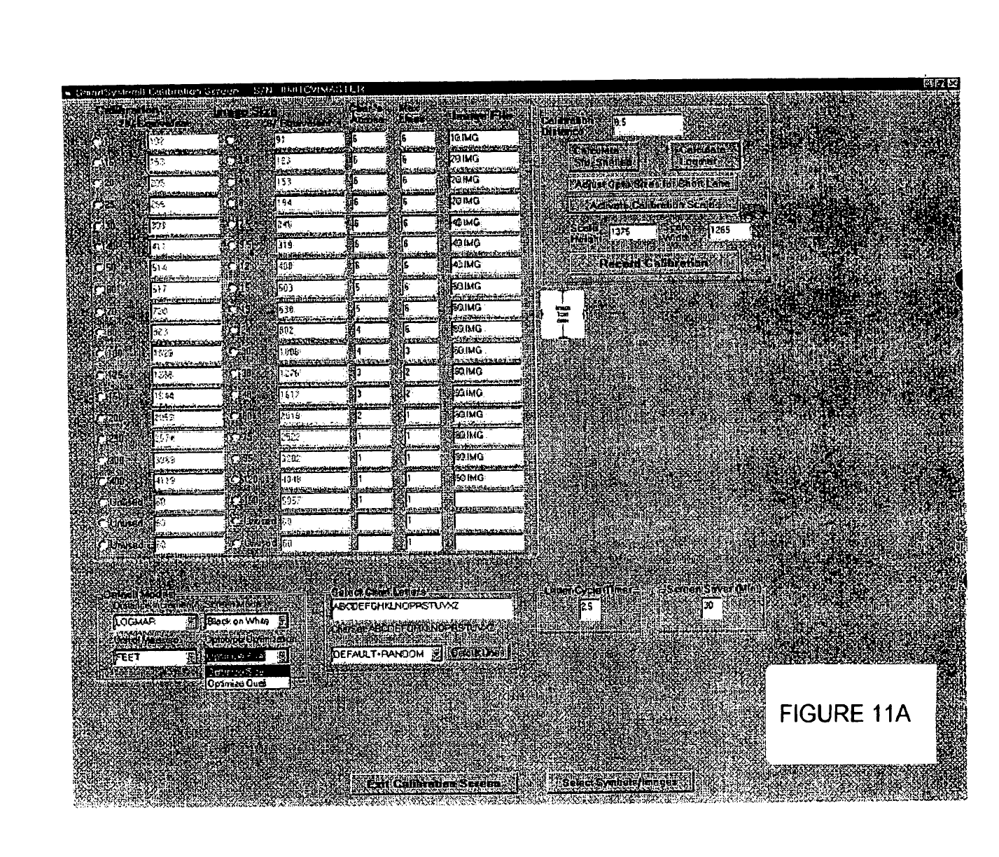

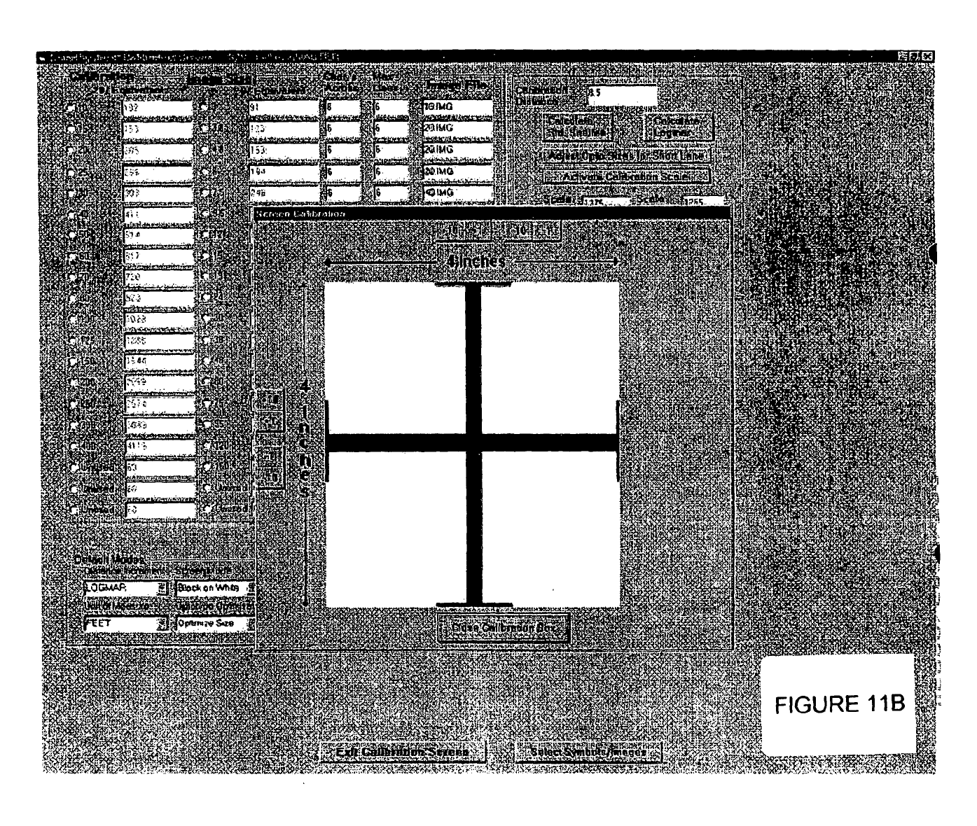

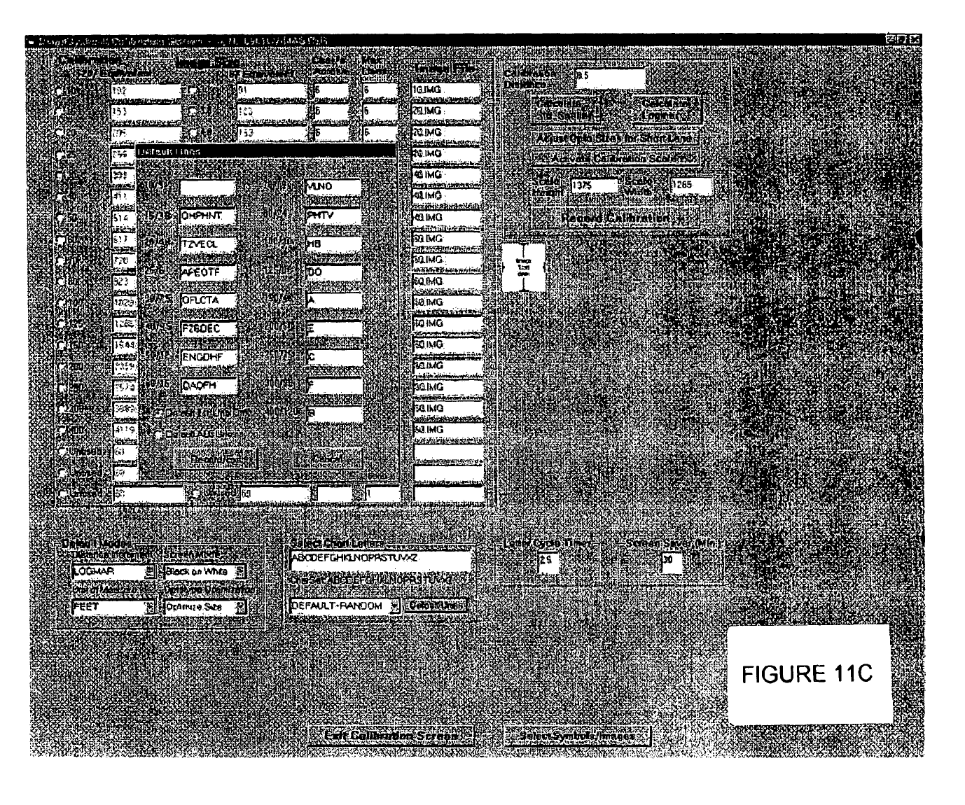

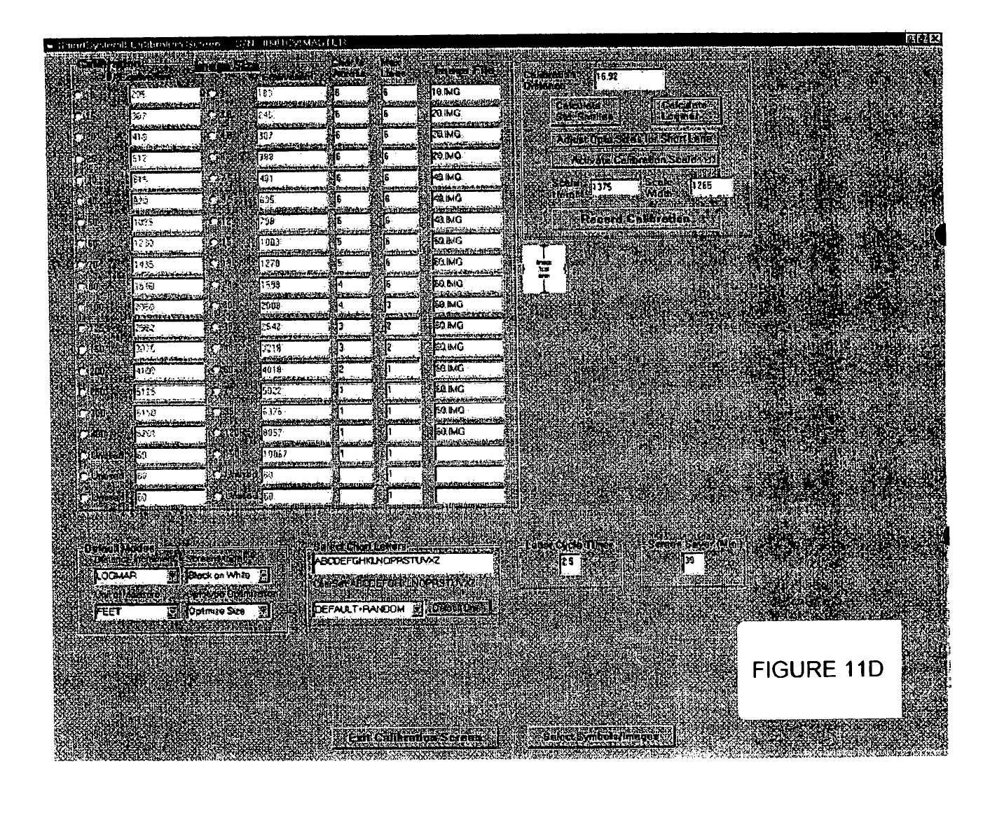

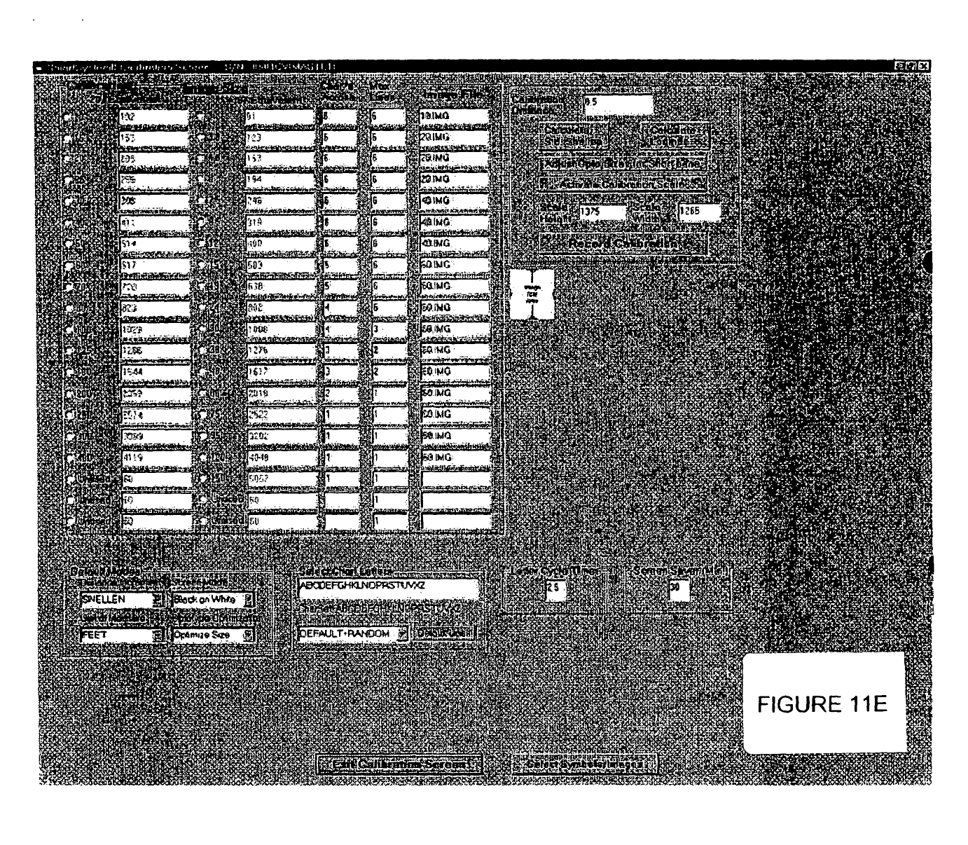

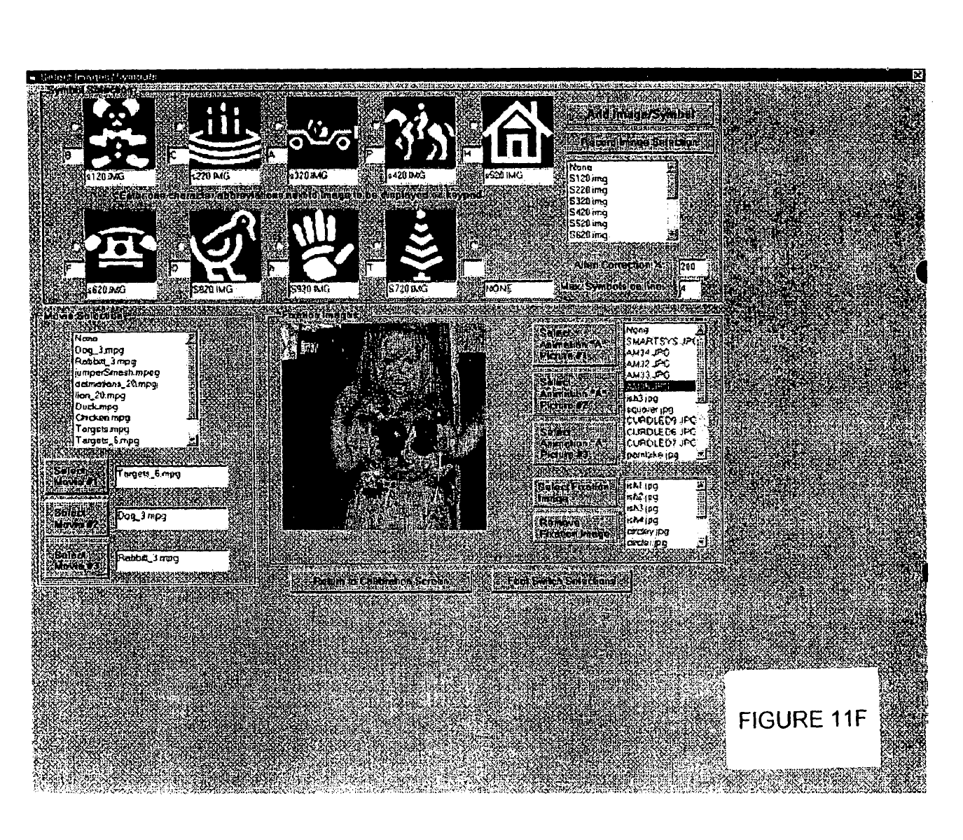

Representative screens are illustrated in FIGS. 11A-11F that allow for customization and calibration of the system. Various data entry boxes, e.g., “Calibration Distance”, “Scale Height”, “Scale Width”, “Distance Increment”, “Screen Mode”, “Unit of Measure”, “Optotype Optimization”, “Select Chart Letters”, “Default Lines”, “Letter Cycle Timer”, and “Screen Saver” (FIG. 1 1A) allow setup information to be input into the VAFCS to customize and calibrate the system. In addition, command buttons, e.g., “Calculate Standard Snellen”, “Calculate Logmar”, “Adjust Opto Sizes for Short Lane”, “Activate Calibration Scale”, “Record Calibration”, “Select Symbols/Images”, (FIG. 11A) and “Close Calibration Box” and “Exit Calibration Screen” (FIG. 11B) allow a user to perform various commands and calibrations by scrolling and clicking on various buttons. Of course, other data entry boxes and command buttons can be used or displayed or in a different fashion without departing from the scope and intent of the present invention.

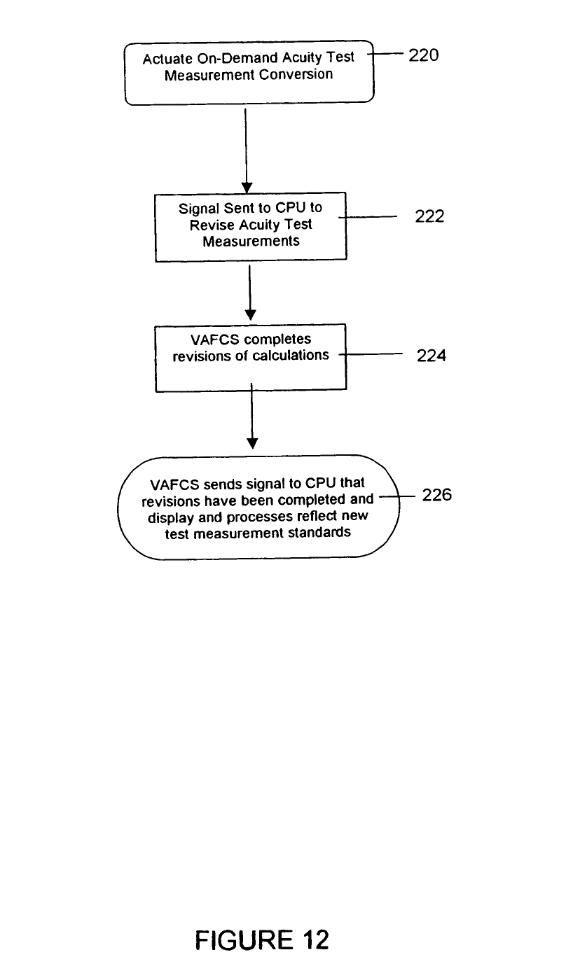

FIG. 12 illustrates one example of a conversion of an acuity test into an alternative measurement standard. Conversion can be initiated by the health care professional depressing the mode key on the keypad controller represented by step 220. The signal to convert the measurements is sent to the CPU, which signals VAFCS to convert the data in step 222. The VAFCS can then convert each of the acuity tests on the dedicated keys into a second standard of measurement in visual acuity in step 224. There are two main standards of measurement within the VAFCS, which are Snellen and Logmar. It is contemplated, however, that the VAT given can be converted to any measurement as is well known. VAFCS then sends a signal to the CPU that the revisions have been completed, and displays and processes that take place from that point forward will reflect the new test measurement standards as represented in step 226.

The invention has been described with reference to the preferred embodiments. Obviously, modifications and alterations will occur to others upon a reading and understanding of this specification. It is intended that the invention be construed as including all such modifications and alterations insofar as they come within the scope of the appended claims or the equivalents thereof.

Having thus disclosed the invention with particularity, the invention is claimed to be:

1. A method for testing the visual acuity of a patient's eyesight comprising the steps of:
displaying a visual acuity test (“VAT”) on a display device;
initiating the VAT via a first controller which is operatively associated with a computer-processing unit (“CPU”); and
fixating the patient's eye on a display device using one of multiple eye fixation assessments stored on the CPU during portions of said visual acuity test initiated by a second controller which is operatively associated with said CPU.

2. The method of claim 1 wherein said displaying step includes outputting data to a computer monitor that is operatively associated with the CPU.

3. The method of claim 1 comprising the further step of displaying which VAT has been initiated by the CPU on a second display device.

4. The method of claim 1 wherein said fixating step includes the steps of foot pedal controller controlling animated fixation targets and independently controlling a video movie from the CPU.

5. The method of claim 1 comprising the further step of calibrating the VAT to a room size.

6. A visual acuity testing apparatus comprising:
a display device;
a computer-processing unit (“CPU”) operatively communicating with the display device;
a first controller operatively associated with said CPU for fixating the patient's eye using an eye fixation assessment on a display device during portions of said visual acuity test; and
a second controller is operatively associated with said CPU for operating which visual acuity examination is initiated by the CPU.

7. The apparatus of claim 6 further comprising an input device adapted to receive data regarding size of an examination room for calibration purposes.

8. The apparatus of claim 6 wherein said CPU includes Visual Acuity and Fixation Control Software (“VAFCS”) that controls display of customized data, images and audio for use in conducting a visual acuity examination.

9. The apparatus of claim 6 wherein said second controller includes a handheld controller.

10. The apparatus of claim 6 wherein said second controller is a handheld controller further including an LCD which displays which visual acuity examination is being initiated by the CPU.

11. The apparatus of claim 6 wherein said second controller is a handheld controller further including phosphorescent keys which indicate which visual acuity test is available for display by the CPU.

12. The apparatus of claim 11 wherein the phosphorescent keys remain bright through the use of light emitting diodes that recharge throughout the day.

13. The apparatus of claim 6 wherein said second controller is a foot pedal controller having multiple switches.

14. The apparatus of claim 5 wherein said foot pedal controller includes three separate controllers used to control animated fixation targets and video movies from the CPU.

15. A method for testing the visual acuity of a patient's eyesight comprising:
initiating a visual acuity test (“VAT”) from a computer-processing unit (“CPU”) via a handheld keypad controller;
displaying said VAT on a display device operatively associated with said CPU; and
conducting an eye assessment using eye fixation images initiated on said display device via a foot control pedal.

16. The method of claim 15 wherein said handheld keypad controller further includes a display which displays which visual acuity examination is being initiated by the CPU.

17. The method of claim 13 wherein said foot pedal controller includes three separate controllers which are used to control animated fixation targets and video movies from the CPU.

18. The method for testing the visual acuity of a patient's eyesight comprising the steps of:
initiating a visual acuity test (“VAT”) via a first controller which is operatively associated with a computer-processing unit (“CPU”);
displaying the VAT on a display device; and
evaluating the contrast sensitivity of the patient's eye with the display device.

19. The method of claim 18 wherein said contrast sensitivity is evaluated by determining the patient's ability to see images of varying contrast relative to the background.

20. The method of claim 19 wherein the patient's ability to see images of varying contrast is determined by altering contrast of the image, background, or both displayed on the display device.

21. The method of claim 18 wherein the contrast sensitivity images of low contrast are set to a zero percentage of the brightness or output intensity or contrast against a white background.

22. The method of claim 20 wherein said image contrast is increased incrementally.

23. The method of claim 22 wherein said incremental increase can be adjusted by as little as 1% increments.

24. The method of claim 22 wherein contrast sensitivity is determined once an incremental amount is reached such that said patient can identify the image.

25. The method of claim 21 wherein said output intensity of the selected image is set to a percentage of brightness or output intensity or contrast relative to the background.

26. The method of claim 20 wherein the contrast of the image is decreased incrementally.

27. The method of claim 26 wherein said image can be decreased in increments of as little as 1%.

28. The method of claim 26 wherein said image intensity is decreased in contrast to the background until said patient can identify the image.

29. The method of claim 18 wherein the contrast sensitivity is evaluated by varying types of images relative to varying types of backgrounds.

30. The method of claim 1820 wherein the contrast sensitivity images of varying contrast are set to a percentage of brightness or output intensity or contrast relative to a background, where the images and backgrounds can be displayed across an infinite spectrum of colors.

31. A method for testing the visual acuity of a patient's eyesight comprising the steps of:
initiating a visual acuity test (“VAT”) from a computer-processing unit (“CPU”) via a handheld keypad controller;
displaying said VAT on a display device operatively associated with said CPU; and
displaying an optotype pointer in conjunction with any single line VAT.

32. The method of claim 31 further comprising the step of selectively moving said optotype from character to character.

33. A method for testing the visual acuity of a patient's eyesight comprising the steps of:
initiating a visual acuity test (“VAT”) from a computer-processing unit (“CPU”) via a handheld keypad controller;
displaying said VAT on a display device operatively associated with said CPU; and
automatic cycling of any single character VAT.

34. A method for testing the visual acuity of a patient's eyesight comprising:
initiating a visual acuity test (“VAT”) from a computer-processing unit (“CPU”) via a handheld keypad controller;
displaying said VAT on a display device operatively associated with said CPU; and
presenting VAT in examination rooms under 10 feet in length.

35. The method of claim 34 comprising the further step of optimizing images through use of one of several graphics resolution files in combination with calculating precise character sizes.
